(19) United States
(12) Patent Application Publication
(10) Pub. No.: US 2021/0018510 A1
(43) Pub. Date: Jan. 21, 2021

(54) MARKERS OF ENDOPLASMIC RETICULUM STRESS IN OVARIAN FOLLICULAR FLUID PREDICT IVF SUCCESS

(71) Applicants: THE REGENTS OF THE UNIVERSITY OF COLORADO, A BODY CORPORATE, Denver, CO (US); WASHINGTON UNIVERSITY, St. Louis, MO (US)

(72) Inventors: Joshua JOHNSON, Greenwood Village, CO (US); Alex J. POLOTSKY, Denver, CO (US); Emily JUNGHEIM, Clayton, MO (US)

(21) Appl. No.: 16/966,827

(22) PCT Filed: Jan. 31, 2019
(86) PCT No.: PCT/US2019/016008

§ 371 Date: Jul. 31, 2020

Related U.S. Application Data
(60) Provisional application No. 62/624,742, filed on Jan. 31, 2018.

(51) Int. Cl. G01N 33/68

(52) U.S. Cl. CPC G01N 33/689

(57) ABSTRACT

The present invention discloses an unbiased “biomarker” method for the prediction of pregnancy success that is detectable in patient follicular fluid (FF) collected during egg retrieval in IVF procedures. Proteins that function in the cellular response to endoplasmic reticulum (ER) stress were measured in FF, and data generated using a de-identified, retrospective, unblinded cohort of human FF and matching demographic and pregnancy outcome data showed that FF levels of ER stress effector proteins GRP78 and CHOP, which are indicative of elevated ER stress in either the oocyte, granulosa cells, or other cells of the follicle, are causally related to poor pregnancy outcomes.

### CROSS REFERENCE TO RELATED APPLICATION

This application claims the benefit of U.S. Provisional Patent Application Ser. No. 62/624,742, filed Jan. 31, 2018, which is incorporated herein by reference.

### TECHNICAL FIELD

This invention relates generally to the fields of reproductive medicine, and biotechnology. More particularly this disclosure relates to the use of protein profiles derived from analysis of ovarian follicular fluid as biomarkers for diagnosis of and/or prognosis for predicting the viability of oocytes for selected biological procedures, especially in vitro fertilization.

### BACKGROUND

Cells undergoing stress, such as glucose deprivation, acidosis, or hypoxia, accumulate underglycosylated and misfolded proteins in the endoplasmic reticulum (ER). Cells under such ER stress employ a highly specific ER quality-control system termed the unfolded protein response (UPR) to restore ER homeostasis (Kim, et al., Nature Reviews Drug Discovery, 7(12):1013-30, 2008). The ability of cells to respond to ER stress is critical for cell survival, and chronic or unresolved ER stress can lead to apoptosis (Tabas, et al., Nature Cell Biology, 13(3):184-90, 2011).

Glucose-regulated protein 78 (GRP78) is an ER molecular chaperone protein that is a major UPR target. Due to its role as a molecular chaperone with anti-apoptotic properties, GRP78 enhances the functional ability of the ER. Conversely, C/EBP homologous protein (CHOP; also known as GADD153), is a pro-apoptotic transcription factor that is also activated during UPR. ER stress-induced cell death is controlled by a number of factors, including a set of pro-apoptotic proteins, among which, CHOP is a key initiating player, and deletion of CHOP protects it against lethal consequences. Thus, GRP78 and CHOP proteins are opposing representatives of the ER stress response.

Currently, selection of the embryos with the highest implantation potential during assisted reproductive technology (ART) procedures relies only on morphological criteria. A method based on time-lapse imaging has been described for the acquisition of embryo morphokinetic data to help with such selection (Herrero and Meseguer, Fertility and Sterility, 99:1030-4, 2103), but this method that relies on subjective observation of embryo morphology to predict a successful pregnancy shows limitations (Aydiner, et al., Current Molecular Medicine, 10:206-15, 2010). Therefore, recent work has focused on identifying biomarkers based on the analysis of the oocyte microenvironment to improve the accuracy of embryo selection. In some studies, follicular fluid (FF) components, which are derived from plasma or secreted from granulosa cells, were investigated as potential biomarkers (Ledee, et al., Human Reproduction 28:406-13, 2013). Indeed, FF, which surrounds the oocyte, is involved in follicular maturation, oocyte growth, and the gradual acquisition of developmental competence. Consequently, FF may represent a reliable source of oocyte and embryo outcome biomarkers that could be used as supplemental prognostic/diagnostic tools in ART.

Therefore, until success rates of ART, in particular in vitro fertilization (IVF), can be improved, it would be desirable to be able to identify recipients for whom ART is unlikely to be successful prior to treatment, so that such patients may avoid the costs and trauma of these procedures.

### SUMMARY

In the present invention, the inventors established an unbiased “biomarker” method for the prediction of pregnancy success that is detectable in patient follicular fluid (FF) collected during egg retrieval in IVF procedures. Proteins that function in the cellular response to endoplasmic reticulum (ER) stress were measured in FF, and data generated using a de-identified, retrospective, unblinded cohort of human FF and matching demographic and pregnancy outcome data showed that FF levels of ER stress effector proteins GRP78 and CHOP, which are indicative of elevated ER stress in either the oocyte, granulosa cells, or other cells of the follicle, are causally related to poor pregnancy outcomes.

This disclosure therefore provides an innovative prognostic test for embryo quality, in which levels of ER stress markers in human follicular fluid, collected at the time of egg retrieval, significantly differ in women and are predictive of women that do or do not achieve positive IVF cycle pregnancy outcomes (serum hCG pregnancy test and live birth).

The inventors found a significant and positive correlation between GRP78 levels in FF and the likelihood of implantation, ongoing pregnancy, and live birth in women undergoing IVF treatment. The inventors also found a significant and negative correlation between CHOP and the likelihood of implantation, ongoing pregnancy, and live birth in women undergoing IVF treatment.

In accordance with the present invention, GRP78 and/or CHOP levels in FF are identified at the time of egg retrieval for diagnostic and prognostic use in assessing egg quality and identifying women undergoing IVF with likely positive or negative pregnancy outcomes.

This disclosure provides methods in which concentrations of GRP78 and/or CHOP are measured in FF and compared with corresponding reference values, thereby providing prediction of successful or unsuccessful outcomes of in vitro fertilization (IVF), including the likelihood of implantation, ongoing pregnancy, and live birth, and to select oocytes which are more likely to lead to a viable pregnancy. Thus, this disclosure provides a more specific method for direct analysis of a reliable source of oocyte and embryo outcome biomarkers that are useful as prognostic/diagnostic tools in ART, and may aid a physician in selecting oocytes that are more likely to result in viable pregnancy, or to modify IVF protocols to obtain, diagnose, or prognose successful outcomes and avoid complications of the therapy or of the procedure as a whole.

This disclosure also provides values of GRP78 and CHOP protein levels and concentrations in FF at the time of egg retrieval from women undergoing IVF, thereby providing methods for assessing egg quality, selecting eggs for successful implantation, and predicting successful or unsuccessful outcomes of in vitro fertilization (IVF), including the likelihood of implantation, ongoing pregnancy, and live birth.

This disclosure also provides kits for determining GRP78 and/or CHOP protein levels in FF for assessing egg quality and predicting successful IVF outcomes, comprising written instructions, at least one internal standard, at least one reference standard, and means to detect GRP78 and/or CHOP proteins in a FF sample. The reference standard(s) may be a quantity or concentration of GRP78 and/or CHOP proteins in FF that is indicative of egg quality or successful IVF procedure.

Thus, this disclosure provides methods, GRP78 and CHOP protein reference levels and concentrations, and kits that allow for identification of patients who are more likely to have a successful or unsuccessful outcome in IVF treatment, for selection of oocytes which are more likely to lead to viable pregnancy following IVF treatment, and the adjustment of IVF treatment regimens to reach the goal of successful ovulation and pregnancy.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF THE DRAWINGS

FIGS. 1A-1C depict the demographics of patients used in the study of IVF success in women with “normal ovarian reserve” (NOR; greater than or equal to 10 eggs retrieved) versus “diminished ovarian reserve (DOR; less than 10 eggs retrieved). FIG. 1A shows a comparison of patient age at time of retrieval. FIG. 1B shows a comparison of patient body mass index (BMI). FIG. 1C shows a comparison of distribution of the number of eggs retrieved in each study group.

FIGS. 2A-2D depict measurements of ER effectors CHOP and GRP78 in FF. FIG. 2A shows that CHOP protein was found to be significantly elevated in the FF of DOR patients compared to NOR patients. FIG. 2B shows a Western blot detection of CHOP (29 kD, *) in two separate FF samples. FIG. 2C shows that GRP78 protein is significantly lower in the FF of DOR patients. (p values, Student's t-test). FIG. 2D shows a Western blot of GRP78 protein (78 kD, *) in two separate FF samples.

FIGS. 3A and 3B show that CHOP protein is significantly elevated in DOR FF samples from women that did not achieve pregnancy. FIG. 3A shows ELISA measurement of CHOP in FF, which revealed that levels were elevated only in DOR samples from women that did not achieve a positive hCG test or live birth. FIG. 3B shows that NOR samples did not show elevation when biochemical pregnancy was not achieved.*** indicates significantly different expression.

FIGS. 4A-4C show the levels of GRP78 alone are not associated with pregnancy outcomes, but ratios of GRP78:CHOP significantly differ between pregnancy outcomes within NOR and DOR groups. FIGS. 4A and 4B show ELISA measurements of GRP78 in FF segregated by by pregnancy outcome. FIG. 4C shows a comparison between the GRP78:CHOP ratio between diagnosis groups and the hCG pregnancy test outcome showed significant differences between negative and positive tests for both groups. This ratio was higher in NOR patients with negative hCG tests and lower in DOR patients with negative hCG tests.

### DETAILED DESCRIPTION

As noted above, methods, reference values, and kits of this disclosure are useful for facilitating assessment oocyte quality, and likelihood of successful in vitro fertilization treatment cycle. The methods and systems can be implemented to, for example, facilitate a determination of success for implantation of embryos, selection of an optimal number of embryos to transfer, avoiding the expense and trauma associated with unsuccessful IVF treatment, and determination of success in subsequent in vitro fertilization treatment cycles following an unsuccessful treatment cycle.

These methods include obtaining GRP78 and/or CHOP protein levels or concentrations in FF samples from a female subject to provide a prognosis of oocyte implantation, ongoing pregnancy, and live birth.

This invention is not limited to particular embodiments described in this disclosure, as such may vary. The terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As used herein and in the appended claims, the singular forms “a”, “an”, and “the” include plural referents unless the context clearly dictates otherwise. Thus, for example, reference to “a cell” includes a plurality of such cells and reference to “the compound” includes reference to one or more compounds and equivalents thereof known to those skilled in the art, and so forth.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

As used herein and in the appended claims, the singular forms “a”, “an”, and “the” include plural reference unless the context clearly dictates otherwise. For example, reference to “a steroid” includes a plurality of such steroids, and reference to the “a steroid profile” is a reference to one or more profiles, and so forth.

As used herein, “comprising,” “including,” “having,” “containing,” “characterized by,” and grammatical equivalents thereof, are inclusive or open-ended terms that do not exclude additional, unrecited elements or method steps, but also include the more restrictive terms “consisting of” and “consisting essentially of.”

As used herein, “successful pregnancy” or “viable pregnancy” means the successful implantation of a fertilized ovum such that fetal development and birth are likely to result.

As used herein, “outcome,” when used in association with “in vitro fertilization,” is inclusive of both viability of an oocyte and non-viability of an oocyte for in vitro fertilization. As used herein, “successful outcome of in vitro fertilization” means successful fertilization of an ovum that is suitable for implantation and intrauterine development.

The terms “subject,” “individual,” and “patient” are used interchangeably herein to refer to a mammal being assessed for treatment and/or being treated. In most instances, the mammal is a female human. The terms “subject,” “individual,” and “patient” thus encompass individuals in need of assessment of oocyte quality and prognosis of success in pregnancy and live birth, including those who have undergone or are candidates for an in vitro fertilization cycle.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

This disclosure relates to an in vitro method for determining the quality of an embryo comprising the steps consisting of measuring the quantity or concentration of CHOP or GRP78 proteins, or both proteins, normalized to total protein content (i.e., the “protein level”), in a sample of FF obtained at the time of oocyte retrieval from a female subject, and comparing the protein level to a reference value to assess the quality of an oocyte retrieved from the subject. In these methods, the step of comparing the protein level to a reference value may also be used to predict the likelihood of successfully establishing pregnancy in the subject using IVF protocols for fertilization and implantation of the retrieved oocyte. In these methods, the step of comparing the protein level to a reference value may also be used to predict the likelihood of successfully achieving live birth from the subject using IVF protocols for fertilization and implantation of the retrieved oocyte.

The term “biological sample” refers to an individual follicular fluid, follicular fluid sample from individual pre-ovulatory follicles, blood, serum, or plasma.

The oocyte may result from a natural cycle, a modified natural cycle or a stimulated cycle for IVF. The term “natural cycle” refers to the natural cycle by which the female produces an oocyte. The term “modified natural cycle” refers to the process by which the female produces an oocyte or two under a mild ovarian stimulation with GnRH antagonists associated with recombinant FSH or hMG. The term “stimulated cycle” refers to the process by which a female produces one or more oocytes under stimulation with GnRH agonists or antagonists associated with recombinant FSH or hMG.

The term “in vitro fertilization” refers to a process by which oocytes are fertilized by sperm outside of the body, in vitro. IVF is a major treatment in infertility when in vivo conception has failed.

Reference to “determining the quality of an oocyte” means determining whether an oocyte is likely competent in the context of in vitro fertilization, including successfully progressing to fertilization, pregnancy, or live birth. Accordingly, the methods of this disclosure allows selection of the best oocyte that is able to give rise to pregnancy.

The term “competent oocyte” refers to an oocyte with a high likelihood of implanting, post fertilization, and leading to pregnancy, and to give rise to a viable fetus, which in turn develops into a viable offspring absent of a procedure or event that terminates the pregnancy.

The method of the invention is applicable preferably to human females but may be applicable to other mammals (e.g., primates, dogs, cats, pigs, cows, or mice).

The term “GRP78” or “CHOP” refer to proteins and also relate to nucleic acids that originate from oocyte or ovarian follicles that go into follicular fluid and/or other biological fluids or compartments.

Any methods well known in the art may be used by the skilled artisan in the art for measuring GRP78 and/or CHOP protein quantities or concentrations in FF, such as a FF sample obtained from a patient. For example, a polyclonal antibody that recognizes dog, mouse, rat, and human GRP78 proteins is commercially available (ThermoFisher Scientific, Rockford, Ill.) and is useful at least in quantifying GRP78 protein in ELISA assay format. Additionally, a human GRP78 ELISA kit is commercially available (LifeSpan Biosciences, Inc., Seattle, Wash.), which is useful for the same purposes. Similarly, rabbit and mouse monoclonal antibodies that recognize human CHOP are commercially available (ThermoFisher Scientific, Rockford, Ill.), and a human CHOP ELISA kit is commercially available (LifeSpan Biosciences, Inc., Seattle, Wash.) that are useful for ELISA and Western blot analysis of CHOP protein levels.

This disclosure provides methods for determining the quality of an oocyte, including the steps of measuring the quantity or concentration of C/-EBP homologous protein (CHOP), normalized to total protein content, in a follicular fluid (FF) sample obtained from a female undergoing in vitro fertilization (IVF) at the time of oocyte harvest, and predicting successful pregnancy of the oocyte if the CHOP protein level is less than or equal to a reference value that is indicative of successful pregnancy. In these methods, the reference value is typically determined from CHOP protein levels found in FF samples from females undergoing IVF treatments that have progressed to successful pregnancy and live birth. Useful and instructive reference values may also be determined from CHOP levels found in FF samples from females that have undergone IVF treatments that did not progress to successful pregnancy and/or live birth.

This disclosure provides methods for determining the quality of an oocyte, including the steps of measuring the quantity or concentration of glucose-regulated protein 78 (GRP78) protein, normalized to total protein content, in a follicular fluid (FF) sample obtained from a female undergoing in vitro fertilization (IVF) at the time of oocyte harvest, and predicting successful pregnancy of the oocyte if the GRP78 protein level is greater than or equal to a reference value that is indicative of successful pregnancy. In these methods, the reference value is typically determined from GRP78 protein levels found in FF samples from females undergoing IVF treatments that have progressed to successful pregnancy and live birth. Useful and instructive reference values may also be determined from GRP78 protein levels found in FF samples from females that have undergone IVF treatments that did not progress to successful pregnancy and/or live birth.

This disclosure also provides methods for determining the quality of an oocyte, including the steps of measuring the quantity or concentration of GRP78 protein and CHOP protein in a follicular fluid (FF) sample, normalized to total protein content, obtained from a female undergoing in vitro fertilization (IVF) at the time of oocyte harvest, calculating the ratio of GRP78:CHOP protein in the FF sample, and predicting successful pregnancy of the oocyte if the GRP78:CHOP ratio is greater than or equal to a reference value that is indicative of successful pregnancy. In these methods, the reference value is typically determined from GRP78:CHOP protein ratios found in FF samples from females undergoing IVF treatments that have progressed to successful pregnancy and live birth. Useful and instructive reference values may also be determined from GRP78:CHOP protein ratios found in FF samples from females that have undergone IVF treatments that did not progress to successful pregnancy and/or live birth.

In the methods of this disclosure, the ovarian reserve status of the female patient may further enhance the reliability of prediction of IVF success, such as positive pregnancy test and/or live birth, when used in conjunction with GRP78 and/or CHOP quantities and concentrations. Diminished ovarian reserve (DOR) is a clinical diagnosis used to describe patients who showed a history of poor response to ovarian stimulation by gonadotropins in controlled ovarian hyperstimulation/intrauterine insemination (COH/IUI) treatment. The diagnosis may be based on poor ovarian response in previous non-IVF infertility treatments, or clomid challenge test, or if tests indicate that they have a low Antral Follicle Count (AFC), a low Anti-Mullerian Hormone (AMH) level, and/or an elevated Follicle Stimulating Hormone (FSH) level. A diagnosis of DOR is highly negatively associated with live birth outcomes in IVF treatments. In the method of this disclosure, the DOR status of a patient is identified by the number of oocytes retrieved at the time of harvest, wherein recovery of less than 10 oocytes is considered a female with DOR, as opposed to a female with normal ovarian reserve (NOR) in which 10 or more oocytes are retrieved at the time of harvest in an IVF treatment cycle. The inventors' research showed that CHOP levels are significantly elevated in all samples from DOR females compared to NOR women, and in samples from DOR patients that did not achieve pregnancy compared to those that did.

Thus, this disclosure provides methods for determining the quality of an oocyte, including the steps of measuring the quantity or concentration of CHOP in a follicular fluid (FF) sample, normalized to total protein content, obtained from a female undergoing in vitro fertilization (IVF) at the time of oocyte harvest wherein fewer than 10 oocytes are retreived, and predicting unsuccessful pregnancy or live birth if the CHOP protein level is greater than or equal to a reference value that is indicative of unsuccessful pregnancy in a DOR female. In these methods, the reference value is typically determined from CHOP protein levels found in FF samples from DOR females that have undergone IVF treatments that did not progress to successful pregnancy and/or live birth.

The inventors' research also showed that GRP78 is present in all FF samples assessed and is significantly reduced in all samples from DOR women compared to NOR women. Thus, this disclosure provides methods for determining the quality of an oocyte, including the steps of measuring the quantity or concentration of GRP78 in a follicular fluid (FF) sample, normalized to total protein content, obtained from a female undergoing in vitro fertilization (IVF) at the time of oocyte harvest wherein fewer than 10 oocytes are retrieved, and predicting unsuccessful pregnancy or live birth if the GRP78 protein level is less than or equal to a reference value that is indicative of unsuccessful pregnancy in a DOR female. In these methods, the reference value is typically determined from GRP78 protein levels found in FF samples from DOR females that have undergone IVF treatments that did not progress to successful pregnancy and/or live birth.

Similarly, this disclosure provides methods for determining the quality of an oocyte, including the steps of measuring the quantity or concentration of GRP78 and CHOP in a follicular fluid (FF) sample, normalized to total protein content, obtained from a female undergoing in vitro fertilization (IVF) at the time of oocyte harvest wherein 10 or more oocytes are retrieved, calculating the GRP78:CHOP ratio, and predicting unsuccessful pregnancy or live birth if the GRP78:CHOP ratio is greater than or equal to a reference value that is indicative of unsuccessful pregnancy in a NOR female. In these methods, the reference value is typically determined from GRP78:CHOP ratios found in FF samples from NOR females that have undergone IVF treatments that did not progress to successful pregnancy and/or live birth. Alternatively, successful pregnancy and/or live birth may be predicted if the GRP78:CHOP ratio is less than or equal to a reference value that is indicative of successful pregnancy in a NOR female. In these methods, the reference value is typically determined from GRP78:CHOP ratios found in FF samples from NOR females that have undergone IVF treatments that did progress to successful pregnancy and/or live birth.

The reference values for CHOP, GRP78, and GRP78:CHOP ratio may be a threshold value or a cut-off value that can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a standard curve based on experimental data. Preferably, the person skilled in the art compares the protein levels in FF (obtained according to the method of the invention) with a defined threshold value. For example, the threshold value may be derived from the protein levels (or ratio, or score) determined in follicular fluid derived from one or more patients undergoing IVF. Furthermore, retrospective measurement of the nucleic acid levels (or ratio, or scores) in properly banked historical follicular fluid of patients undergoing IVF may be used in establishing these threshold values.

In embodiments of the methods of this disclosure, the reference value for CHOP protein concentration in a FF sample, expressed as a ratio to normalized total protein content in the FF sample, that is associated with or indicative of unsuccessful pregnancy (i.e., negative pregnancy test) in a DOR female undergoing IVF treatment is about 18 or greater.

In embodiments of the methods of this disclosure, the reference value for CHOP protein concentration in a FF sample, expressed as a ratio to normalized total protein content in the FF sample, that is associated with or indicative of successful pregnancy (i.e., positive pregnancy test) in a DOR female undergoing IVF treatment is about 14 or less.

In embodiments of the methods of this disclosure, the reference value for CHOP protein concentration in a FF sample, expressed as a ratio to normalized total protein content in the FF sample, that is associated with or indicative of unsuccessful IVF outcome (i.e., no resulting live birth) in a DOR female undergoing IVF treatment is about 17 or greater.

In embodiments of the methods of this disclosure, the reference value for CHOP protein concentration in a FF sample, expressed as a ratio to normalized total protein content in the FF sample, that is associated with or indicative of successful IVF outcome (i.e., live birth) in a DOR female undergoing IVF treatment is about 13 or less.

In embodiments of the methods of this disclosure, the reference value for GRP78 protein concentration in a FF sample, expressed as a ratio to normalized total protein content in the FF sample, that is associated with or indicative of unsuccessful pregnancy (i.e., negative pregnancy test) in a normal ovarian reserve (NOR) female undergoing IVF treatment is about 800 or greater.

In embodiments of the methods of this disclosure, the reference value for GRP78 protein concentration in a FF sample, expressed as a ratio to normalized total protein content in the FF sample, that is associated with or indicative of successful pregnancy (i.e., positive pregnancy test) in a normal ovarian reserve (NOR) female undergoing IVF treatment is about 500 or less.

In embodiments of the methods of this disclosure, the reference value for GRP78 protein concentration in a FF sample, expressed as a ratio to normalized total protein content in the FF sample, that is associated with or indicative of unsuccessful IVF treatment (i.e., no live birth) in a normal ovarian reserve (NOR) female undergoing IVF treatment is about 450 or greater.

In embodiments of the methods of this disclosure, the reference value for GRP78 protein concentration in a FF sample, expressed as a ratio to normalized total protein content in the FF sample, that is associated with or indicative of successful IVF treatment (i.e., live birth) in a normal ovarian reserve (NOR) female undergoing IVF treatment is about 300 or less.

In embodiments of the methods of this disclosure, the reference value for GRP78:CHOP ratio in a FF sample, that is associated with or indicative of unsuccessful pregnancy (i.e., negative pregnancy test) in a normal ovarian reserve (NOR) female undergoing IVF treatment is about 4×10⁴ or greater.

In embodiments of the methods of this disclosure, the reference value for GRP78 protein concentration in a FF sample that is associated with or indicative of successful pregnancy (i.e., positive pregnancy test) in a normal ovarian reserve (NOR) female undergoing IVF treatment is less than 4×10⁴.

Determination of the quantity or concentration of the proteins (GRP78 and CHOP) and total protein content can be performed by a variety of techniques well known in the art. In exemplary embodiments, the protein levels in FF samples may be determined by ELISA assay techniques described in the examples.

These reference values may correspond to the protein level determined in a biological sample such as follicular fluid associated with a competent embryo. Accordingly, a higher or equal protein level of CHOP than the reference value is indicative of lower likelihood of successful IVF treatment (positive pregnancy test or live birth) in NDOR females, and a lower protein level of CHOP than the reference value is indicative of higher likelihood of successful IVF treatment in DOR females.

These reference values may correspond to the protein level determined in a biological sample such as follicular fluid associated with a competent embryo. Accordingly, a higher or equal protein level of GRP78 than the reference value is indicative of lower likelihood of successful IVF treatment (positive pregnancy test or live birth) in NOR females, and lower protein level of GRP78 than the reference value is indicative of higher likelihood of successful IVF treatment in NOR females.

These reference values may correspond to the protein level determined in a biological sample such as follicular fluid associated with a competent embryo. Accordingly, a higher or equal GRP78:CHOP ratio than the reference value is indicative of lower likelihood of successful IVF treatment (positive pregnancy test or live birth) in NOR females, and lower GRP78:CHOP ratio than the reference value is indicative of higher likelihood of successful IVF treatment in NOR females.

The methods of this disclosure are helpful for reaching a clinical decision during IVF treatment. As used herein the term “clinical decision” refers to any decision to take or not take an action that has an outcome that affects the health, or fertilization, or implantation of the oocyte. In particular, in the context of the methods of this disclosure, a clinical decision refers to a decision to implant or not implant the embryo of in the uterus of the female patient. These methods will thus help an embryologist to avoid the transfer in uterus of embryos with a poor potential for pregnancy outcome. These methods may also improve in vitro fertilization outcomes and avoidance of multiple pregnancies by selecting the competent embryo able to lead to an implantation and a pregnancy and therefore fewer embryos could be transferred at each cycle, resulting in a decreased incidence of multiple pregnancies.

In a further aspect, the invention relates to a method for enhancing the pregnancy outcome of a patient comprising the steps consisting of i) providing a plurality of embryos, ii) determining the quality of the embryo by the method according to the invention, iii) selecting the most competent embryo, and iv) implanting the embryo selected at step iii) in the uterus of said patient.

A method of implanting a competent embryo in a patient undergoing in vitro fertilization, comprising the steps of: a) collecting oocytes from the patient; b) determining the quality of the embryo by performing the methods of this disclosure described above; c) generating embryos from said oocytes by fertilizing said oocytes in vitro; and d) implanting said embryo having a higher probability of being competent in said patient.

This disclosure also relates to kits for performing the methods as described above. These kits comprise means for determining the CHOP and/or GRP78 protein concentrations. Typically, the kits include anti-CHOP and/or anti-GRP78 antibodies. The kit may further comprise hybridization reagents or other suitably packaged reagents and materials needed for the detection or quantification protocols, including solid-phase matrices, if applicable, and standards. The antibodies may be pre-labelled or may contain an affinity purification or attachment moiety.

The methods of this disclosure will be further illustrated by the following figures and examples. However, these examples and figures should not be interpreted in any way as limiting the scope of the present invention.

### EXAMPLES

### Example 1: Methods for the Analysis of Steroid Patterns in FF Samples from RM Women Participants

To determine/detect endoplasmic reticulum (ER) stress in FF, the inventors generated data using a de-identified, retrospective, unblinded cohort of human FF and matching demographic and pregnancy outcome data. Based on the demonstration that the ER stress marker GRP78 can be secreted by cells of the lung to extracellular space (Aksoy et al., Respir. Res., 18(1):78, 2017), a retina model system [Zhongiie et al., Am J Clin Nutr. 101:879-88, 2015) where free fatty acid levels corresponded to ER stress, and our own prior findings (Al-Safi et al, JCEM, 101:324-333, 2016), the inventors hypothesized that FF levels of ER stress effector proteins GRP78 and CHOP would be detectable in patient FF.

To test this hypothesis, FF from the lead follicle was collected during egg retrievals from female patients undergoing IVF treatment, as previously described (Jungheim et al., Fertil. Steril. 95:1970-74, 2011). Following collection, the FF samples may be frozen at −80° C., and thawed before analyzed by ELISA assay or western blot. Samples were categorized as DOR when fewer than 10 eggs were retrieved and “normal reserve” (NOR) when greater than or equal to 10 eggs were retrieved (n=12 samples per group). The ER stress markers C/EBP homologous protein (CHOP) and GRP78/BiP were measured by commercial ELISA assays, and Western blot analysis was used to confirm detection of bona fide CHOP protein. Data were reported as the ratios between the concentration of each protein and total FF protein, and were segregated by biochemical pregnancy (positive hCG test) and live birth outcomes.

Prior to western blot or ELISA, the total protein concentration for each follicular fluid sample is determined using a small aliquot and a commercial BCA assay.

For western blotting, a volume of follicular fluid corresponding to 10, 20, or 30 micrograms is added to western sample buffer and boiled prior to gel electrophoresis and following the attached protocol through imaging bands on the membrane. Western blots have been used as qualitative positive control confirmation of the presence of CHOP and separately, GRP78, in human follicular fluid.

For ELISA analysis, either 100 microliters of straight, undiluted follicular fluid (for CHOP measurement) or 100 microliters of follicular fluid diluted 1:5 (GRP78 measurement) are added to each well in the plate in triplicate. In each plate, a standard curve of positive control protein concentrations is included and used to calculate the “unknown” concentrations of CHOP and GRP78 in our follicular fluid samples.

The statistical programming environment R within Rstudio was used for all analysis of ELISA data. Plate reader data was generated directly to a Microsoft Excel spreadsheet, and raw data were saved. Data from individual experiments were exported to comma-delimited .csv file format and imported into Rstudio. Patient data were segregated according to diagnostic criteria of ovarian reserve, a clinical feature that the inventors hypothesized might relate to levels of ER stress markers. The ratio of CHOP or GRP78 to total sample protein was calculated within R, and data were segregated according to pregnancy outcomes (positive hCG pregnancy test and separately, live birth) by patient group (DOR vs. NOR). Student's t-test was used to determine statistical significance between the levels of CHOP, GRP78, or, the GRP78:CHOP ratio by pregnancy outcome within patient groups, and summary data and plots were produced within Rstudio. All raw data, unedited but rearranged data (into .csv format), and code used to produce these analyses are available upon request for reproducibility purposes.

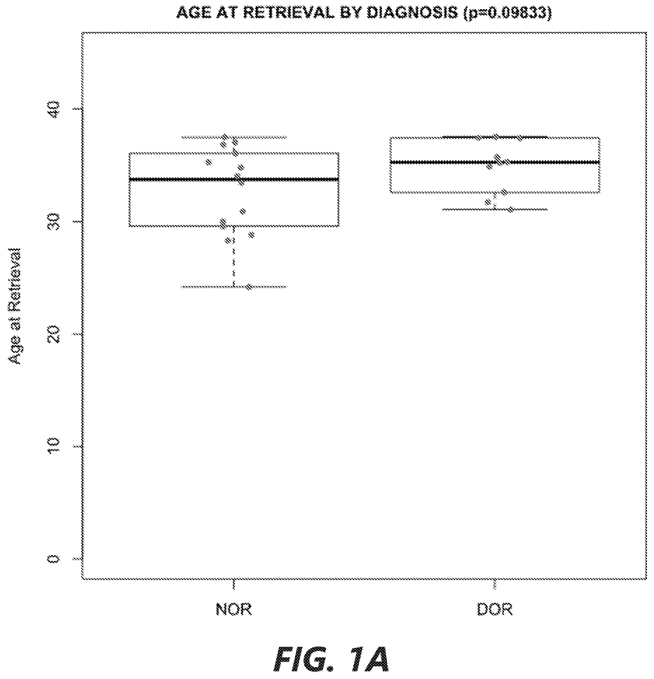

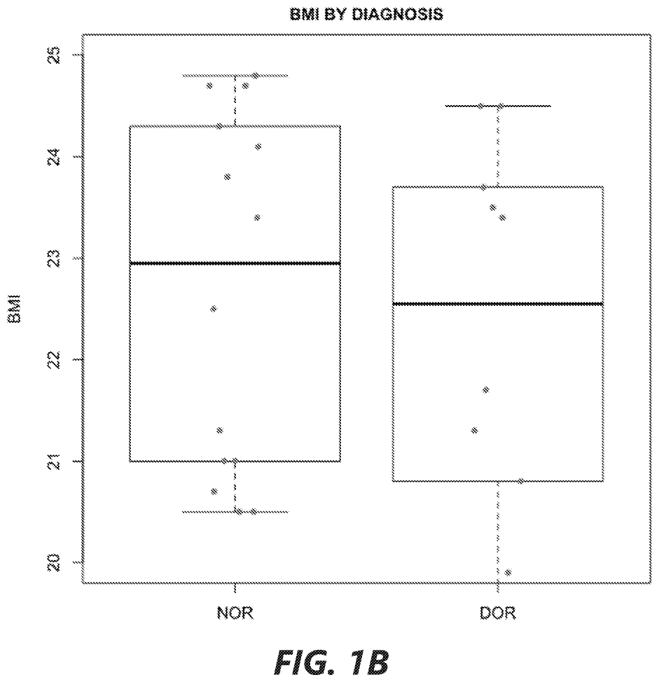

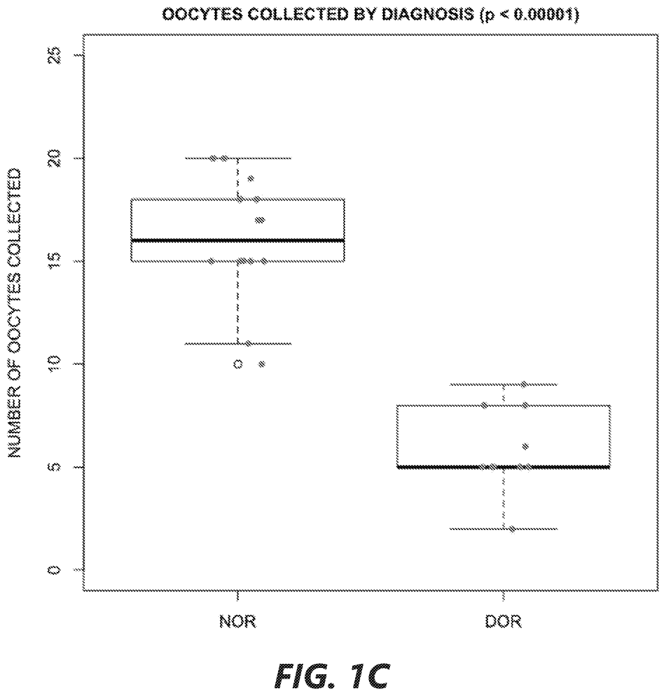

FIGS. 1A-1C depict the demographics of patients used in the study of IVF success in women with “normal ovarian reserve” (NOR; greater than or equal to 10 eggs retrieved) versus “diminished ovarian reserve (DOR; less than 10 eggs retrieved). FIG. 1A shows a comparison of patient age at time of retrieval. FIG. 1B shows a comparison of patient body mass index (BMI). FIG. 1C shows a comparison of distribution of the number of eggs retrieved in each study group.

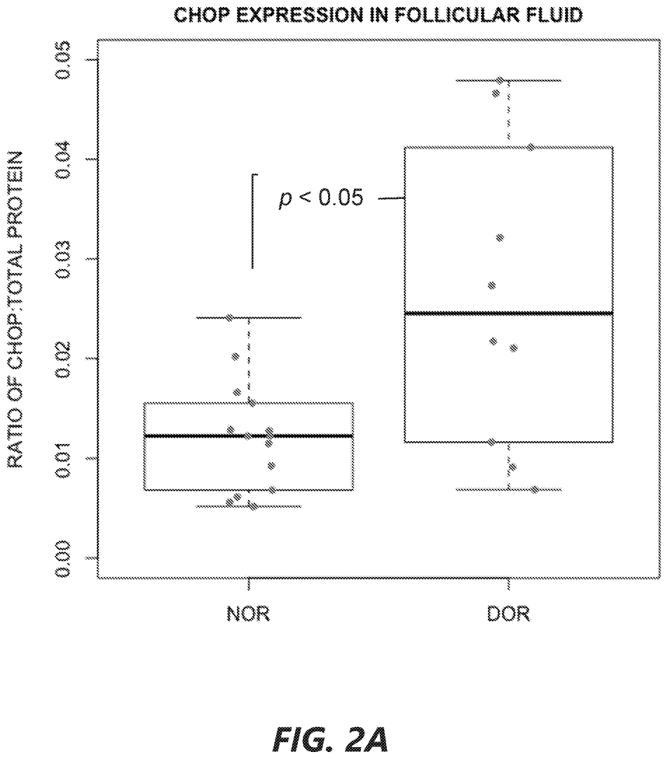

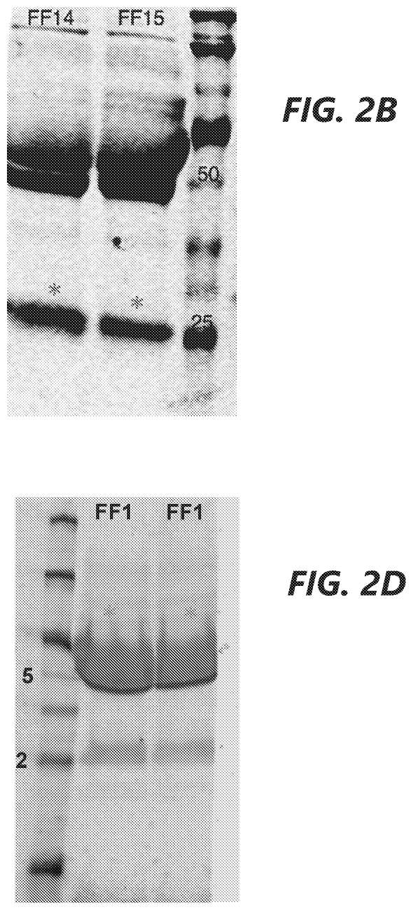

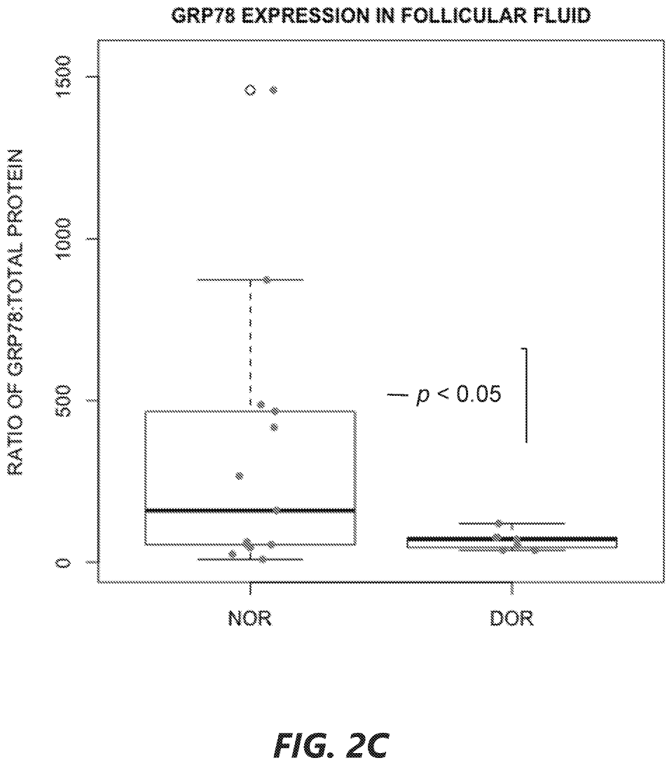

FIGS. 2A-2C depict measurements of ER effectors CHOP and GRP78 in FF. FIG. 2A shows that CHOP protein was found to be significantly elevated in the FF of DOR patients compared to NOR patients. FIG. 2B shows a Western blot detection of CHOP protein (29 kD, *) in two separate FF samples. FIG. 2C shows that GRP78 protein is significantly lower in the FF of DOR patients. (p values, Student's t-test).

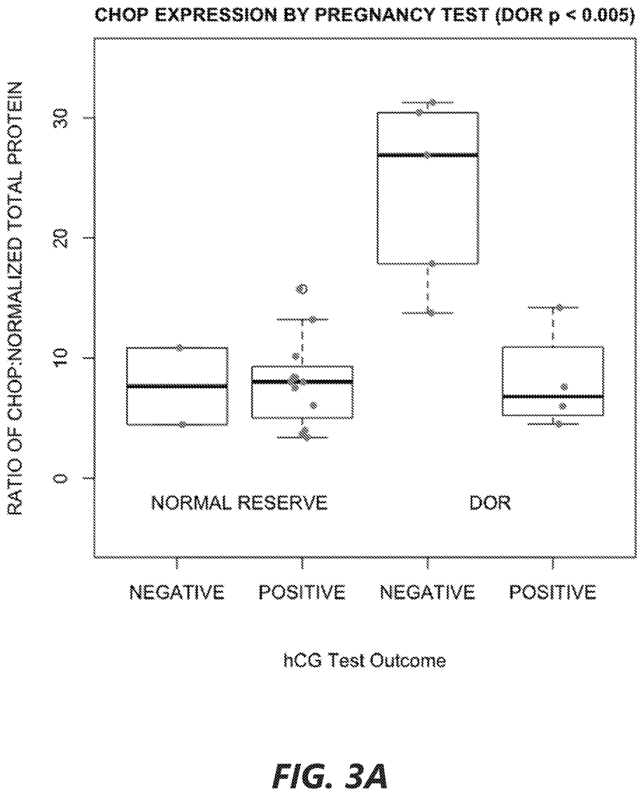

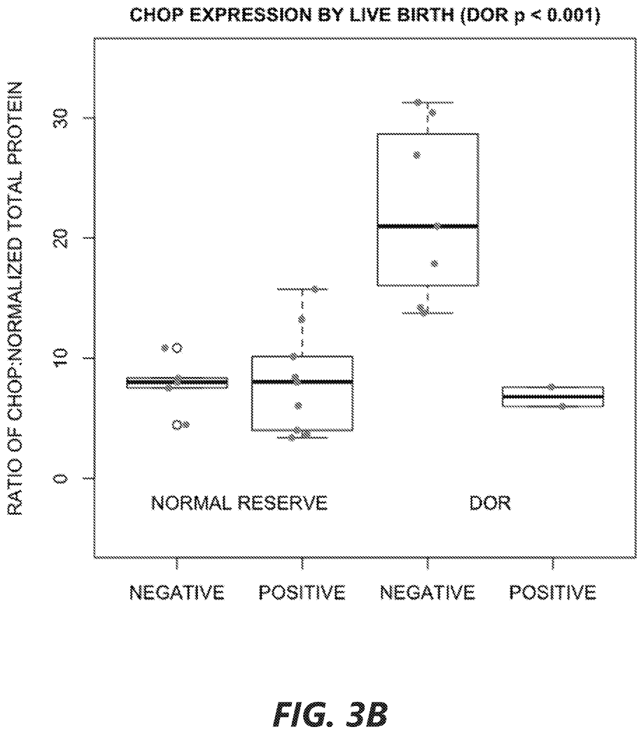

FIGS. 3A and 3B show that CHOP protein is significantly elevated in DOR FF samples from women that did not achieve pregnancy. FIG. 3A shows ELISA measurement of CHOP in FF, which revealed that levels were elevated only in DOR samples from women that did not achieve a positive hCG test or live birth. FIG. 3B shows that NOR samples did not show elevation when biochemical pregnancy was not achieved.*** indicates significantly different expression.

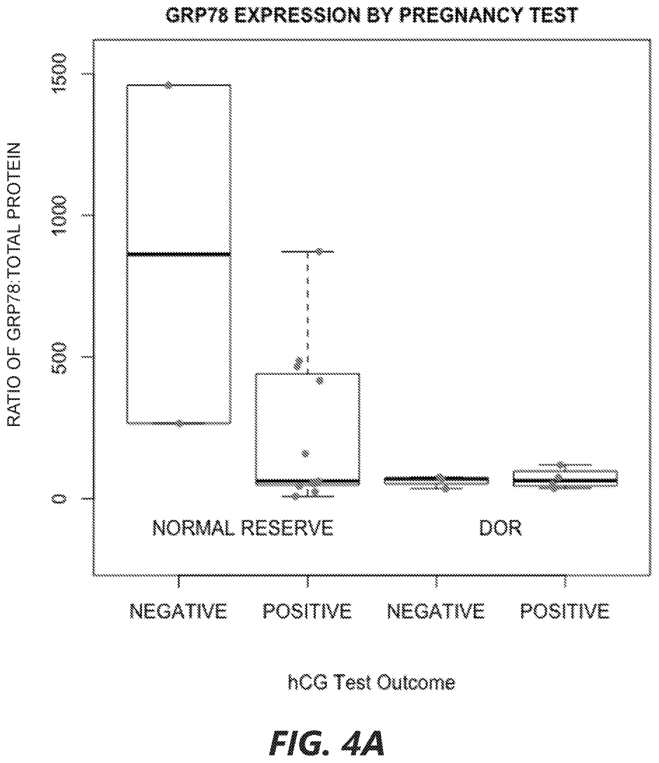

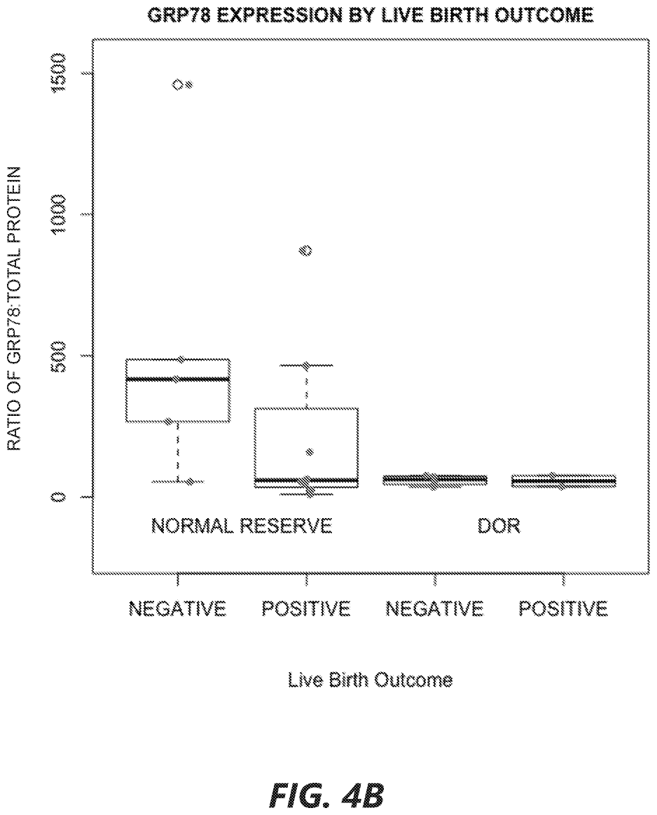

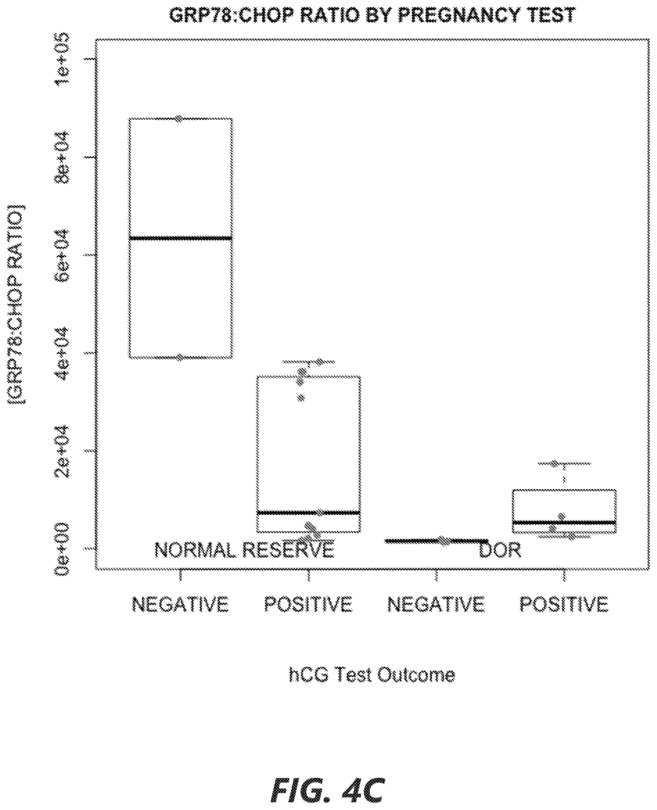

FIGS. 4A-4C show the levels of GRP78 alone are not associated with pregnancy outcomes, but ratios of GRP78:CHOP significantly differ between pregnancy outcomes within NOR and DOR groups. FIGS. 4A and 4B show ELISA measurements of GRP78 in FF segregated by by pregnancy outcome. FIG. 4C shows a comparison between the GRP78:CHOP ratio between diagnosis groups and the hCG pregnancy test outcome showed significant differences between negative and positive tests for both groups. This ratio was higher in NOR patients with negative hCG tests and lower in DOR patients with negative hCG tests.

Collectively, these data demonstrate that CHOP is present in all FF samples and is significantly elevated in FF samples from DOR women compared to NOR women, and also in samples from DOR patients that did not achieve pregnancy compared to those that did. GRP78 is present in all samples assessed and is significantly reduced in all samples from DOR women compared to NOR women. Thus, predictive information is available at the time of egg retrieval (the time of CHOP and GRP78 measurement) about pregnancy outcomes for DOR and NOR patients.

These data suggest that ER stress may be both a FF biomarker of the likelihood of implantation and ongoing pregnancy and potentially, and a causal factor in egg quality that impacts pregnancy outcomes.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

1. An in vitro method for determining the quality of an oocyte comprising the steps of:
i) measuring the quantity or concentration of C/-EBP homologous protein (CHOP),
normalized to total protein content, to obtain a CHOP protein level in a follicular fluid sample obtained from a female undergoing in vitro fertilization (IVF) at the time of oocyte harvest, and
ii) predicting successful pregnancy of the oocyte if the CHOP protein level is below a reference value that is indicative of successful pregnancy.

2. The method of claim 1 further comprising the steps of:
i)
ii) measuring the quantity or concentration of glucose-regulated protein 78 (GRP78) protein, normalized to total protein content, in the follicular fluid sample,
iii) calculating the ratio of the GRP78:CHOP protein levels in the FF sample, and
iv) predicting successful pregnancy of the oocyte if the ratio of the GRP78:CHOP protein level ratio is above a reference value that is indicative of successful pregnancy.

3. The methods of claim 1, wherein fewer than 10 oocytes are harvested from the female at the time of oocyte harvest.

4. The methods of claim 1, wherein 10 or more oocytes are harvested from the female at the time of oocyte harvest.

5.-6. (canceled)

7. An in vitro method for predicting successful or unsuccessful pregnancy in IVF treatment comprising the steps of:
i) measuring the quantity or concentration of CHOP in a follicular fluid sample, normalized to total protein content, obtained from a female undergoing in vitro fertilization (IVF) at the time of oocyte harvest wherein 10 or more oocytes are retrieved,
ii) measuring the quantity or concentration of GRP78, normalized to total protein content, in the follicular fluid sample,
iii) calculating a GRP78:CHOP ratio
iv) predicting unsuccessful pregnancy of the oocyte in the female if the GRP78:CHOP ratio is greater than a reference value that is indicative of unsuccessful pregnancy or
v) predicting successful pregnancy of the oocyte in the female if the GR78:CHOP is below a reference value that is indicative of successful pregnancy.

8.-10. (canceled)

11. The method of claim 1, wherein a successful pregnancy is determined as at least one of a positive serum hCG pregnancy test and a live birth.

12. The method of claim 1, wherein the quantity or concentration of the protein is measured by enzyme-linked immunosorbent assay (ELISA) or Western blot.

13. The method of claim 1, wherein the FF sample is isolated from the first follicle retrieved from the female.

14. The methods of claim 1, wherein the measuring step comprises measuring the protein concentration within total protein content of the FF sample.

15. (canceled)

16. A method of providing a prognosis for in vitro fertilization treatment or outcome, the method comprising:
i) measuring the quantity or concentration of at least one protein selected from GRP78 and CHOP, normalized to total protein content, from a sample of ovarian follicular fluid from a female;
ii) evaluating the level of the at least one protein in comparison with one or more reference values characteristic of an outcome of IVF; and
iii) providing a prognosis of a selected outcome in the female based on the evaluation.

17. The method of claim 16, wherein providing the prognosis of a selected outcome comprises determining that an oocyte is more likely to result in a positive serum hCG pregnancy test.

18. The method of claim 16, wherein the selected outcome is the prognosis of likely
viability or non-viability of oocytes for a successful in vitro fertilization outcome.

19. The method of claim 16, wherein providing the prognosis of a selected outcome comprises determining that an oocyte is more likely to result in a live birth.

20. The method of claim 16, wherein the quantity or concentration of the protein is measured by enzyme-linked immunosorbent assay (ELISA) or Western blot.

21. The method of claim 16, wherein 10 or more oocytes are harvested from the female at the time of oocyte harvest.

22. The method of claim 16, wherein fewer than 10 oocytes are harvested from the female at the time of oocyte harvest.

23. The method of claim 16, wherein the FF sample is isolated from the first follicle
retrieved from the female.

24. The method of claim 16, wherein the measuring step comprises measuring the protein concentration within total protein content of the FF sample.

25. The method of claim 16, wherein the prognosis of a selected outcome in the female is a prediction of successful pregnancy if the GRP78 protein level is above a
reference value that is indicative of successful pregnancy following the use of an oocyte from an FF sample from a female with a successful pregnancy.

26. The method of claim 16, wherein the prognosis of a selected outcome in the female is a prediction of successful pregnancy if the CHOP protein level is below a reference value that is indicative of successful pregnancy following the use of an oocyte from
an FF sample from a female with a successful pregnancy.
